(19) United States
(12) Patent Application Publication
(10) Pub. No.: US 2012/0220835 A1
(43) Pub. Date: Aug. 30, 2012

(54) WIRELESS PHYSIOLOGICAL SENSOR SYSTEM AND METHOD

(76) Inventor: Wayne Chung, San Francisco, CA (US)

(21) Appl. No.: 13/371,464

(22) Filed: Feb. 12, 2012

Related U.S. Application Data
(60) Provisional application No. 61/442,600, filed on Feb. 14, 2011.

(51) Int. Cl. A61B 5/0205; A61B 5/01; A61B 5/11; A61B 5/053; A61B 5/0408

(52) U.S. Cl. 600/301

(57) ABSTRACT

Embodiments of the present invention relate generally to wireless medical monitoring. In particular, some preferred embodiments of the present invention provide a wearable compact body sensor capable of wireless data transmission to a mobile internet platform. The body sensor includes a plurality of sensors including, for example, a temperature sensor, a heart rate sensor, a respiratory rate sensor, an impedance sensor, an electrocardiogram (ECG) sensor, and a ballistocardiogram (BCG) sensor. The physiological data collected by the body sensor can be sent to or accessed by a physician or health care provider.

### CROSS-REFERENCE TO RELATED APPLICATIONS

This application claims the benefit of U.S. Provisional Application No. 61/442,600 filed Feb. 14, 2011, titled “Wireless Physiological Sensor System and Method,” which is hereby incorporated by reference in its entirety.

### BACKGROUND OF THE INVENTION

1. Field of the Invention

Embodiments of the present invention relate generally to wireless medical monitoring. In particular, some preferred embodiments of the present invention provide a wearable compact body sensor patch capable of wireless data transmission to a mobile internet platform.

2. Description of the Related Art

Currently, there are over one million patient discharges annually in the United States for congestive heart failure (CHF) and a similar amount of patient discharges for arrhythmias, post myocardial infarction, post-stent placement and post-cardiac bypass surgery.

Approximately 20 percent to 30 percent of these patient discharges are re-admitted to a hospital within 30 days after discharge, with great morbidity and/or mortality to the patient, and a cost burden of nearly $10 billion to the health care system. The causes of the readmissions often include, for example, behavior noncompliance by the patient, such as not following up with the primary care doctor, not filling a medication prescription, and not adhering to a medication and diet regimen. Other causes of readmissions include, for example, inherent physiological changes in patients such as new arrhythmias and ischemias.

Active, remote electrocardiogram based telemonitoring has been shown to reduce mortality by up to 30% and readmissions by approximately 40%. Current setups, however, are costly because construction and maintenance of call centers are usually required, and bulky, expensive, complex, non-disposable equipment is needed for patient monitoring, requiring patient and nurse training. The cost for these remote telemonitoring services often outweigh the potential cost savings in reduced re-admissions.

Accordingly, it would be desirable to provide a next generation, low cost sensor and mobile software system to remotely monitor these patients post-discharge.

### SUMMARY OF THE INVENTION

In some embodiments, a wearable body sensor for remotely monitoring a patient is provided. The body sensor includes a case having a skin contact side configured to contact the patient's skin. The case encloses a microcontroller, a battery configured to power the microcontroller, writable memory configured to store data collected by the body sensor, a wireless transmitter in communication with the microcontroller, an impedance sensor in communication with the microcontroller, an electrocardiogram sensor in communication with the microcontroller, a ballistocardiogram sensor in communication with the microcontroller, a patient orientation sensor in communication with the microcontroller, and at least two electrodes attached to the skin contact side of the case.

In some embodiments, the wearable body sensor includes a case that is flexible and water-resistant.

In some embodiments, the wearable body sensor further includes a temperature sensor, wherein the temperature sensor is enclosed at least partly by the case.

In some embodiments, the wearable body sensor further includes a heart rate sensor, wherein the heart rate sensor is enclosed at least partly by the case.

In some embodiments, the wearable body sensor further includes a patient orientation sensor, wherein the patient orientation sensor is enclosed at least partly by the case and includes an accelerometer.

In some embodiments, the wearable body sensor further includes an adhesive disposed on the skin contact side of the case.

In some embodiments, a remote patient monitoring system is provided. The remote patient monitoring system includes a wearable body sensor. The body sensor includes a case having a skin contact side configured to contact the patient's skin. The case encloses a microcontroller, a battery configured to power the microcontroller, writable memory configured to store data collected by the body sensor, a wireless transmitter in communication with the microcontroller, an impedance sensor in communication with the microcontroller, an electrocardiogram sensor in communication with the microcontroller, a ballistocardiogram sensor in communication with the microcontroller, a patient orientation sensor in communication with the microcontroller, and at least two electrodes attached to the skin contact side of the case. A first cellular device is in wireless communication with the body sensor.

In some embodiments, the remote patient monitoring system includes a computer in communication with the first cellular device.

In some embodiments, the remote patient monitoring system includes a second cellular device in wireless communication with the computer.

### BRIEF DESCRIPTION OF THE DRAWINGS

FIG. 1 illustrates an embodiment of a body sensor with its component parts.

FIG. 2 illustrates an embodiment of a body sensor with an adhesive patch as viewed from the skin contact side.

FIG. 3 illustrates an embodiment of a remote patient monitoring system.

FIG. 4 illustrates a flow diagram of measuring, storing, processing and transmitting impedance, electrocardiogram, and ballistocardiogram data.

### DETAILED DESCRIPTION OF THE PREFERRED EMBODIMENT

Preferred embodiments of the present invention provide a low cost body sensor and mobile software system to remotely monitor patents post-discharge. In particular, the body sensor can perform an electrocardiogram, a ballistocardiogram, and/or impedance measurement.

Examples of current remote monitoring systems include U.S. Publication No. 20070225611, U.S. Publication No. 20070249946, and U.S. Publication No. 20070255153 to Kumar et al., which are hereby incorporated by reference in their entireties. Other examples include U.S. Publication No. 20090073991 to Landrum et al., U.S. Publication No. 20090076345 to Manicka et al., and U.S. Publication No. 20090076350 to Bly et al., which are hereby incorporated by reference in their entireties.

The electrocardiogram provides useful information regarding cardiac function by measuring the electrical activity of the heart, and is especially useful for detection of cardiac rhythm abnormalities caused by or associated with a variety of diseases and pathologies, including for example, myocardial infarction, heart murmurs, cardiac dysrhythmias, syncope, seizures, and critically ill patients.

The ballistocardiogram measures the minute movement of the patient's body in response to the ejection of blood from the heart and into the aorta, and provides a second, independent measurement of cardiac function. The ballistocardiogram provides information that can be correlated to cardiac output, cardiac force, and ejection velocity.

Cardiac or thoracic impedance measurements provide a third, independent source of information on cardiac function by measuring the changes in impedance in the patient's thorax which occurs as the result of changes in the volume of blood in the patient's aorta that occurs with each heartbeat. Impedance measurements also provide information that can be correlated to cardiac output. Abnormal impedance measurements also can signal fluid accumulation in the lungs, which is often a sign of congestive heart failure.

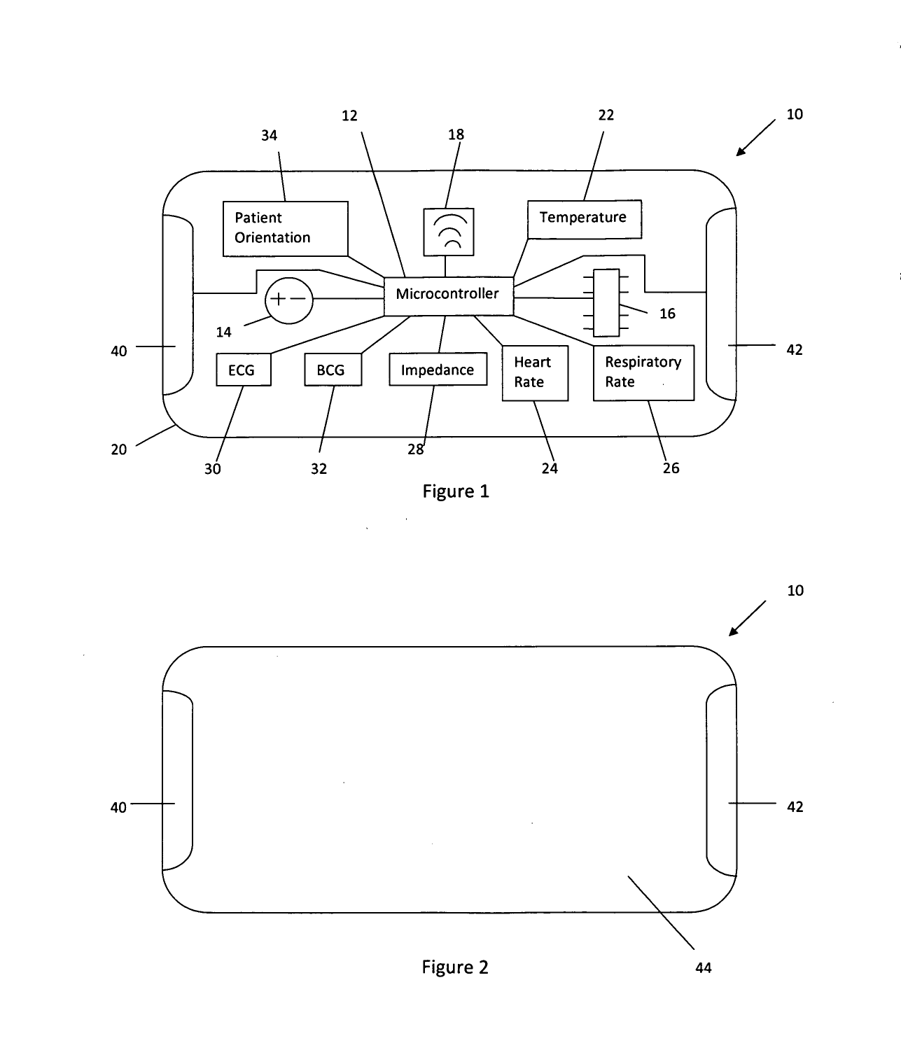

FIG. 1 illustrates an embodiment of a body sensor 10 that can be worn on the chest near or around the lower end of the sternum like a bandage. The body sensor 10 includes a microcontroller 12, a battery 14, writable memory 16, a wireless transmitter 18, and a plurality of sensors, all in a flexible, water-resistant form factor 20 or case. The plurality of sensors can include, for example, a temperature sensor 22, a heart rate sensor 24, a respiratory rate sensor 26, an impedance sensor 28, an electrocardiogram (ECG) sensor 30, a ballistocardiogram (BCG) sensor 32, a patient orientation sensor 34, a hydration sensor 36, and/or an echocardiogram sensor 38. The microcontroller, ECG sensor, and other sensors can be purchased from, for example, Freescale Semiconductor, Inc. and Texas Instruments, Inc.

An accelerometer can be used to detect the periodic chest wall movements from respiration, thereby measuring the respiratory rate and forming the basis of the respiratory rate sensor 26. An accelerometer can also be used to measure the minute movements of the patient's body from the ejection of blood from the heart and into the aorta, thereby forming the basis of the BCG sensor 32. Further description regarding the BCG sensor can be found in, for example, U.S. Pat. No. 7,846,104 to MacQuarrie et al., which is hereby incorporated by reference in its entirety. An accelerometer can also be used to measure the patient's orientation, i.e., whether the patient is lying down or sitting up or standing, thereby forming the basis of the patient orientation sensor 34. In some embodiments, a single accelerometer can be used by more than one sensor. In other embodiments, a sensor can have a dedicated accelerometer.

The hydration state of the patient can be correlated with the impedance measurement because the hydration state is related to the extracellular fluid status of the patient, and changes in the amount of extracellular fluid results in corresponding changes in impedance measurements.

In some embodiments, the body sensor 10 includes at least two electrodes 40, 42, where the electrodes 40, 42 are positioned on opposing ends of the longer dimension of the skin contact side of the body sensor 10. The two electrodes 40, 42 can be used to record a one lead ECG waveform. The electrodes 40, 42 can also be used to measure impedance and hydration, for example, and other vital signs. Alternatively, additional dedicated electrodes can be added to the body sensor 10 to measure impedance and the other vital signs. The electrodes 40, 42 can be made from a conductive material, such as metal, for example.

In some embodiments, the body sensor 10, wireless transmitter 18, and/or microcontroller 12 and sensors can be ultra-low power or low power, which allows the body sensor 10 to be worn continuously by the patient for over about 24 hours, 48 hours, 72 hours, 96 hours, 120 hours, 144 hours, or 168 hours, or between about 7 to 14 days, or over about 14 days for example.

In some embodiments, the wireless transmitter 18 can be a Bluetooth transmitter. In other embodiments, the wireless transmitter 18 can be a Wi-Fi transmitter, a wireless USB transmitter or a cellular transmitter. In some embodiments, the battery 14 can be an alkaline battery, a lithium battery, a lithium ion battery, a nickel metal hydride battery, a nickel cadmium battery, a disposable or non-rechargeable battery, or a rechargeable battery.

The microcontroller 12 includes embedded software that allows the microcontroller 12 to detect abnormal or potentially abnormal ECG rhythms, BCG rhythms, impedance measurements, and/or other sensor readings, either independently or in combination. Abnormalities or potential abnormalities can be determined by comparing a sensor reading to a known baseline standard, which can be generated from a population of healthy people, unhealthy patients with a similar condition or medical problem, from the patient himself, and/or from published information. The different physiological signals, such as heart rate, respiration rate, ECG signals, BCG signals, impedance signals, fluid status, patient activity data, and temperature for example, recorded by the various sensors can be weighted and combined to determine an index that associates physiological parameters to an impending adverse event such as cardiac decompensation, for example.

As illustrated in FIG. 2, in some embodiments the body sensor 10 has an adhesive coating 44 on the back of the body sensor 10 which makes contact with the patient's skin. In some embodiments, the adhesive coating 44 is permanently attached to the body sensor 10. In other embodiments, the adhesive coating 44 can be an adhesive patch that is attached to the back of the body sensor 10 and that can be removed from the body sensor 10 and replaced with a new adhesive patch when desired. In some embodiments, the adhesive coating 44 and/or the body sensor 10 are water resistant or waterproof. In some embodiments, the adhesive coating 44 remains sticky and/or tacky for at least about 24 hours, 48 hours, 72 hours, 96 hours, 120 hours, 144 hours, or 168 hours, or between about 7 to 14 days, or over about 14 days for example.

In some embodiments, the adhesive coating 44 is capable of conducting electrical signals from the patient's skin to the electrodes 40, 42 of the body sensor 10. In some embodiments, electrodes 40, 42 can be embedded in the adhesive coating 44 to make direct contact with the patient's skin. In some embodiments, the adhesive coating 44 coats the skin side of the body sensor 10 but does not coat the electrodes 40, 42, thereby allowing the electrodes 40, 42 to directly contact the skin. In some embodiments, the adhesive patch that is attached to the skin side of the body sensor 10 has openings for the electrodes 40, 42, such that the electrodes 40, 42 can still make direct contact with the patient's skin after the adhesive patch is applied to the skin side of the body sensor 10.

In some embodiments, the body sensor 10 is affixed to the patient's chest with a strap. The strap can be used in conjunction with the adhesive coating 44, or be used instead of the adhesive coating 44.

In some embodiments, the electrodes 40, 42 can extend away from the body sensor 10 on wires that are connected to the body sensor 10. The electrodes 40, 42 can be embedded in small, discrete adhesive pads that allow flexible placement of the electrodes 40, 42 on the patient's body. In some embodiments, the body sensor 10 can have 3 or more electrodes, some of which are fixed to the casing of the body sensor 10 and some of which are on wires extending from the body sensor 10. In some embodiments, the body sensor 10 can have 3 or more electrodes that are all on wires extending from the body sensor 10. In some embodiments, the electrodes on wires can be plugged into electrode ports on the body sensor 10 when needed, allowing the body sensor 10 have a variable number of electrodes which can be tailored to the needs of the patient.

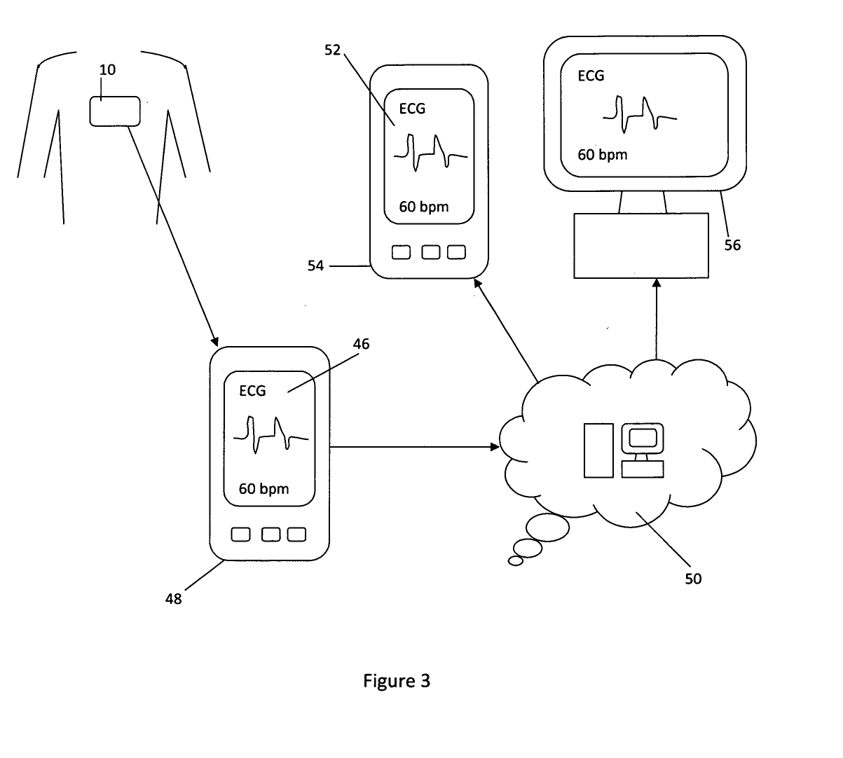

FIG. 3 illustrates an embodiment of the body sensor 10 on a patient wirelessly transmitting physiological data to a mobile application 46 on a cellular device 48. The cellular device 48 can be a mobile phone or a smartphone, such as an iPhone, Android phone, Windows phone, or a Blackberry phone, for example. The body sensor 10 can use any suitable wireless transmission protocol, such as Bluetooth, Wi-Fi, or wireless USB for example, to transmit the physiological data. The cellular device 48 then sends the physiological data to a server, workstation, or computer on the cloud 50, i.e., internet. The cellular device 48 can send the physiological data to the cloud 50 via any acceptable protocol, such as for example, Wi-Fi or a cellular data communication protocol like GSM, CDMA, EV-DO, WiMAX, LTE, 3G, 4G, or the like. The cloud 50 then sends the physiological data to a mobile application 52 on a cellular device 54 of the patient's physician or health care provider using, for example, a cellular data communication protocol like GSM, CDMA, EV-DO, WiMAX, LTE, 3G, 4G, or the like. In addition or alternatively, the cloud 50 can send the physiological data to a computer 56, such as workstation, personal computer, notebook computer, laptop computer, tablet computer, PDA, or the like, of the patient's physician or health care provider. In addition, the physician or health care provider can access the physiological data by logging onto a secure website that stores, analyzes and presents the patient's data to the physician or health care provider. The physician or health care provider can access this website using a cellular device 48, for example, or a workstation, personal computer, notebook computer, laptop computer, tablet computer, PDA, or the like. The physician or health care provider can view the raw data, filtered data, or processed data, such as the ECG and BCG signals, for example.

In some embodiments, the body sensor 10 transmits the physiological data directly to the cloud 50 via a standard wireless protocol such as Wi-Fi or a cellular data communication protocol like GSM, CDMA, EV-DO, WiMAX, LTE, 3G, 4G, or the like. Such as system eliminates the need for a cellular device 48 to be carried by or located near the patient.

Before transmission of the data to a cellular device 48, the body sensor 10 stores the data on the writable memory 16. In the absence of any adverse event, or under standard conditions, the body sensor can transmit data periodically in bursts to the mobile application 46. By transmitting data periodically in bursts, rather than transmitting data continuously, power consumption by the body sensor 10 can be reduced, thereby extending the operational time that the device can be used before the battery 14 must be recharged or replaced. Periodic transmission can occur approximately every 1 minute, 5 minutes, 10 minutes, 15 minutes, 20 minutes, 30 minutes, 40 minutes, 50 minutes, 60 minutes, 2 hours, 3 hours, 4 hours, 5 hours, 6 hours, 7 hours, 8 hours, 9 hours, 10 hours, 11 hours, or 12 hours.

If the body sensor 10 detects a potential abnormality or adverse event, the body sensor 10 can be triggered to immediately transmit the physiological data to the mobile application 46. For example, if the body sensor 10 measures a rapid heart rate of greater than 100 beats per minute in combination of a respiratory rate indicative of a resting state, then the patient may be presenting with symptoms of tachycardia, an adverse event which causes the body sensor 10 to transmit the physiological data to the mobile application 46. In addition, the body sensor can transmit an alarm signal on the detection of a potential abnormal and/or adverse event, which can trigger the mobile application to notify the patient's doctor, the hospital, medical personnel, EMTs, an emergency call center and/or other caregivers or health industry workers. Other abnormal and/or adverse events include, for example, a slow heart rate substantially less than the normal rate, an abnormal QRS complex measured by the ECG, and high body temperature above 37 degrees Celsius.

Digital signal processing can be used to reduce the noise from the physiological data. For example, respiration causes periodic movement of the chest, which can interfere with BCG measurements, which rely on minute movements of the patient's torso. An average adult at rest takes approximately 12 to 20 breaths per minute, for example. During exercise, an adult takes approximately 35 to 70 breaths per minute. The respiratory rate also varies by age. For example, infants average approximately 40 to 60 breaths per minute at rest, preschool children average approximately 20 to 30 breaths per minute, and older children average approximately 16 to 25 breaths per minute. The heart rate, which is also monitored by the body sensor 10, occurs at a substantially quicker rate. For example, an adult at rest has a normal heart rate between approximately 60 to 80 beats per minute. Like the respiratory rate, the heart rate increases with activity, and also is higher in infants and children. Because the rates of respiration differs from the heart rate and the movement of the chest during respiration is much larger than the movements caused by the ejection of blood from the heart, the movement of the chest during the respiratory rate can be recorded and isolated from the minute movement of the torso caused by the ejection of blood into the aorta during a heart beat, resulting in a cleaner BCG signal. The heart rate signal and ECG signals can both be used as timing references to identify, isolate and extract the BCG signal from the respiratory signal. Similarly, the body sensor 10, which has an accelerometer, can detect when a patient is moving, such as walking or running, and digital signal processing techniques can be used to isolate these movement activities from the BCG measurement. If such activities cause too much noise for the BCG measurement, then the BCG measurements can be temporarily halted until these activities cease and normal BCG measurements can again be taken. The most accurate data collection generally occurs, with respect to at least BCG measurements, when the patient is still, like for example, when the patient is sleeping. This time, which is generally also night time, is also frequently when most sudden cardiac events occur.

In some embodiments, some digital signal processing can be done on the body sensor 10 by the microcontroller 12 or by a separate digital signal processing module. In some embodiments, some digital signal processing can be done on the mobile application 46 on the cellular device 48. In some embodiments, some digital processing can be done on the cloud 50. In some embodiments, some digital processing can be done by the computer 56 or mobile application 52 on the cellular device 54 of the physician or health care provider.

Because more computing power is located on the cloud 50 than on the body sensor 10 or cellular device 46, the computationally intensive digital signal processing tasks can be more advantageously be done on the cloud 50. Reducing the amount of digital signal processing done on the body sensor 10 also helps extend the battery 14 life.

Physiological data can be stored on the cloud and be used to develop or update an algorithm to detect, diagnose or predict an impeding adverse event, such as an adverse cardiac event. The algorithm can then be incorporated into the mobile application 46, 52 on the cellular device 46, 54. In some embodiments, the output of the algorithm can be simply a yes or no for an impeding adverse event. In some embodiments, the output of the algorithm can be a risk factor or probability factor for an impeding adverse event.

In some embodiments, the algorithm can combine changes in impedance and fluid status data and changes in cardiac output as determined from BCG data with changes in ECG data to determine high risk periods for an impeding adverse event. For example, a lowered impedance reading may signal increased fluid in the lungs, a sign of congestive heart failure. Decreased cardiac output from the BCG data would also be consistent with symptoms of congestive heart failure. Abnormal ECG readings can further provide more evidence of congestive heart failure. In some embodiments, for each positive signal of an impending adverse event, the risk of an adverse event is increased. In some embodiments, the physician or health care provider is notified when at least one signal indicates a risk of an adverse event. In some embodiments, the physician or health care provider is notified when at least two signals indicate a risk of an adverse event. In some embodiments, the physician or health care provider is notified when at least three signals indicate a risk of an adverse event.

In some embodiments, the physiological data is encrypted and transmitted securely at all stages in compliance with all health information privacy laws. Access to the physiological data is provided to patients, physicians and/or other health care providers and entities that have legal access to such information. The mobile application and/or web access to the physiological data can be password protected and/or be secured using other methods, such as a biometric scan using a retinal scan, a fingerprint scan, or a palm scan and the like.

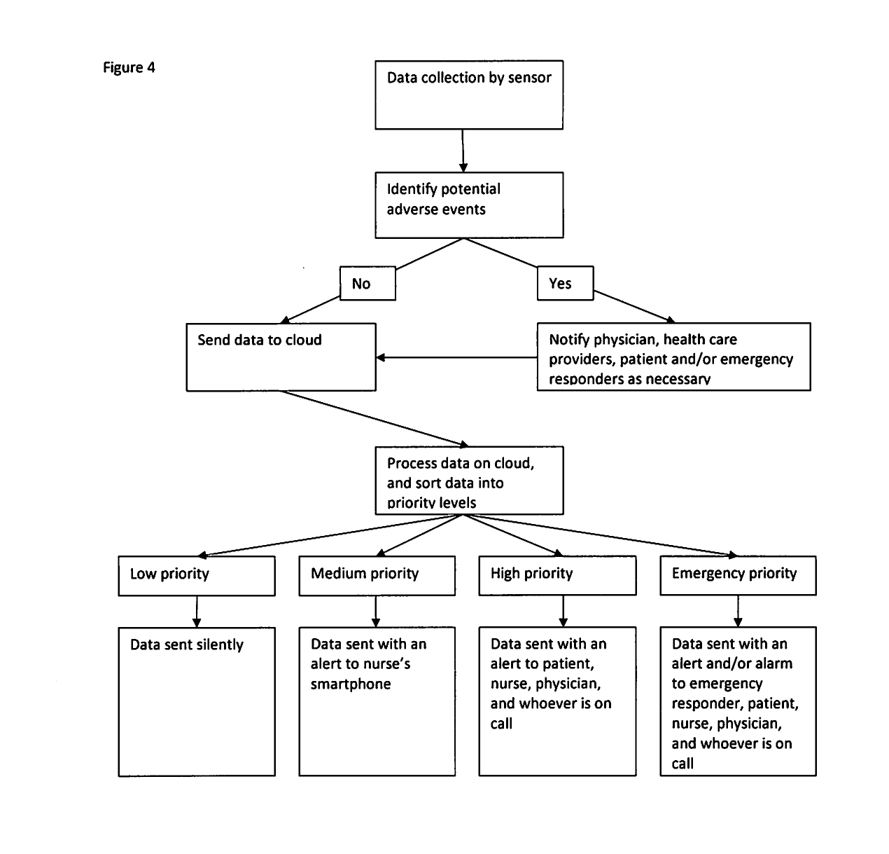

FIG. 4 illustrates a flow chart that shows how physiological data is routed to various health care providers in some embodiments. Physiological data is collected by the body sensor 10. An algorithm on the body sensor 10 can analyze the data and identify potential adverse events. If no potential adverse event is detected, the data is sent to the cloud 50. If a potential adverse event is detected, the body sensor 10 will first initiate a routine to notify the physician, health care providers, patient and/or emergency responders as necessary, and then send the data to the cloud 50. The data is then processed on the cloud 50 and sorted into priority levels. For example, the data can be sorted into a low priority level, a medium priority level, a high priority level, and an emergency priority level. Lowest priority data can be sent silently without an alert to, for example, a call center, and/or an ECG technician's or other health care provider's smartphone. Medium priority data can be sent real-time with an alert to a nurse's smartphone or to another health care provider's smartphone who is responsible for the daily monitoring of the patient. High priority data can be real-time with an alert to the smartphone of a patient, physician, nurse, and/or health care provider who is on call. Emergency priority data can be sent directly with an alert and/or alarm to emergency responders, as well as to the patient, nurse, physician and whoever is on call. Although alerts and/or alarms are provided to physicians and other health care providers, the ultimate diagnosis and treatment remains in the hand of the patient's physician and other health care providers.

In some embodiments, the physiological data can be integrated into a mobile electronic health/medical record (mEHR) on the mobile application 46 on the cellular device 48, for example, and/or the mEHR can be compiled together and accessed on the cloud 50 from a cellular device 48, a workstation, personal computer, notebook computer, laptop computer, tablet computer, PDA, or the like. The mEHR can be integrated with and/or compiled from the electronic health/medical records (EHR) and personal health records (PHR) of patients at clinics, hospitals, and the like, using, for example, Health Level Seven (HL-7) industry standards or another suitable industry standard. The mEHR can include demographic data such as the patient's address, phone, fax, email, emergency contacts, caregiver contacts. The mEHR can also include medical data including current and past medication lists, allergies, insurance provider and coverage, preferred pharmacy, labs, and hospitals.

The mobile application 46 on the cellular device 48, which can also be ported onto the cloud 50 and onto a workstation, personal computer, notebook computer, laptop computer, tablet computer, PDA, or the like, can have a user interface that incorporates both an intuitive text and voice activated interface. In addition, video-conferencing capabilities can be integrated into the mobile application 46 and related software on the cloud 50 so that the physician or health care provider can virtually evaluate a patient and e-prescribe and e-bill afterwards.

The mobile application 46 and cellular device 48 platform allows the physician, nurse, or health care provider to view real-time sensor data, past sensor data, recent and past laboratory results, current and past medication lists, x-rays, MRIs, CAT scans, ultrasounds and other medical images , and any other information contained in the patient's mEHR and PHR. The physician can contact the patient by, for example, calling, emailing, voice mailing, or texting the patient, and then document the contact with the patient using the mobile application 46 and also perform charting and ordering medications, labs, or a further personal visit or appointment, all with the relevant coding of diseases, signs and symptoms, abnormal findings, complaints, social circumstances and external causes of injury or diseases, such as ICD-10 coding, and reimbursement. The patient can also access the data on their PHR or mEHR, thus improving patient empowerment and compliance. The PHR and/or mEHR can include a provider list which includes, for example, caregivers, physicians, nurses, social works, and other health care providers. The mobile health monitoring solution will make the physician workflow easier, more timely, and ultimately be reimbursable with cost savings for all parties.

In some embodiments, the physiological data and eMHR on the mobile application 46 and related software are securely protected from unauthorized access in compliance with all health information privacy laws. For example, access by the patient, physician, or health care provider, requires a login and password. The login and password can be conventionally entered using text, or can be entered using one's voice. In addition, access can be secured using biometrics such as voice recognition, facial recognition, thumb scans, palm scans, and/or retinal scans, for example. In addition, once the patient is logged on, the mobile application 46 can direct the body sensor 10 to automatically begin taking readings.

The mobile application 46 can also utilize the camera on the cellular device 48 to take photographs and videos of a patient presenting physically observable symptoms, such as pallor, sweatiness, and/or tremors, for example. These photographs and videos can be time stamped and notated by the patient, physician and/or health care provider and be used to compare baseline appearance with subsequent appearance during or after, for example, an adverse event such as cardiac decompensation. Symptoms can be identified and compared using pattern recognition software to, for example, an index or databank of previous patients at various degrees of severity.

The mobile application 46 and related software can include map software or access to online maps such as Google Maps, as well as GPS functionality. The location of the patient, pharmacies, laboratories, physician offices, urgent care, emergency room, and/or hospitals, can be given to the patient, emergency responders, caregivers, and physicians, for example, along with turn-by-turn directs.

The mobile application 46 and related software can take, store, and organize data taken by the body sensor 10 and collected from the eMHR and PHR. For example, current ECG and BCG rhythm data, impedance data, fluid status data, temperature, heart rate, respiratory rate, patient activity data can be stored, organized, and displayed along with data from the last one hour, 2 hours, 4 hours, 8 hours, 12 hours, 24 hours, 7 days, or 30 days, or any time period up to 30 days, or any time period up to 2, 3, 4, 5, 6, 7, 8, 9, 10, 11, or 12 months. Any new data can be incorporated into the patient's eMHR and PHR.

As discussed above, the mobile application 46 and related software can send clinical alerts that can be tiered based on priority level to a call center, nurse, physician, and/or emergency responder, for example. In addition, equipment alerts can be sent to the nurse, physician or technician, for example, when the body sensor 10 is dislodged or malfunctioning, or when the wireless transmission is impaired, or when the GPS functionality is impaired, or when the battery is low, or when maintenance is required.

The mobile application 46 can provide physicians, nurses, and other health care providers videoconferencing capabilities, voice/text dictation capabilities, e-prescribing of medications, coding and billing, active patient census, previous patient census, and sign out capabilities for health care providers such as physicians, nurses, technicians, case workers and the like.

In some embodiments, the mobile application 46 can be integrated with the EHR and PHR through HL-7 standards. New data acquired by the body sensor 10 along with entries and input entered in by health care providers such as nurses, physicians, technicians and case workers, can be integrated with the patient's EHR and mEHR. For example, a physician can remotely evaluate a patient via a videoconference, record the videoconference with a patient, make entries during the videoconference regarding the patient's status and treatment plan, and have the mobile application 46 automatically update the EHR and PHR, which can be done real-time.

The mobile application 46 can be integrated with the patient's social network platform, such as Facebook, for example, so that selected family, relatives and friends can receive updates, progress reports, and edited health data real-time and in summary about the patient, thereby decreasing isolation and encouraging behavioral compliance and improving the patient's well-being. The updates can be sent via email or text or Facebook mail, for example, and can contain goals and reminders for the patient to, for example, get medication, take medication, contact and/or see their doctor, or perform weight measurements. This feature allows the selected family, relatives, and friends to encourage and monitor the patient's compliance to the prescribed medical treatment plan. The updates can also provide selected family relatives, and friends the option of sending the patient a gift, which can be either real or virtual. For example, the update can provide the option to purchase real flowers, a real card, virtual flowers, or an e-card. These updates are especially useful for frequent and/or long term care patients, where caregivers can suffer from burnout.

In some embodiments, the mobile application 46 allows the patient to purchase additional services from the health care provider, such as the physician, nurse, or technician, for example. For example, the patient can purchase increased telephone, cell phone, email, videoconference access, and scheduling priority with the physician or other health care provider for a fee. The physician, nurse or other health care provider can elect which additional services they are willing to provide and for what cost. In some embodiments, certain features of the mobile application 46 can be unlocked for a fee. For example, access to the mEHR, EHR or PHR can be provided for a fee. Similarly, any feature described above can be provided for a fee.

In some embodiments, the mobile application 46 can connect with a pharmacy or medical supplier, such as an online pharmacy or medical supplier, to purchase medications and medical supplies. The mobile application 46 can contain drug prescription and renewal information, which can be shared with the pharmacy or medical supplier. The mobile application 46 can include a medication list, both past and current, and include the side effects of those medications, including any adverse drug interactions. The mobile application 46 can take all the side effects and weight each side effect according to frequency and severity while taking into account factors such as sex, race, weight and age, and then sort, list and present the side effects according to their weighted score.

The various devices, methods and techniques described above provide a number of ways to carry out the invention. Of course, it is to be understood that not necessarily all objectives or advantages described may be achieved in accordance with any particular embodiment described herein. Also, although the invention has been disclosed in the context of certain embodiments and examples, it will be understood by those skilled in the art that the invention extends beyond the specifically disclosed embodiments to other alternative embodiments and/or uses and obvious modifications and equivalents thereof. Accordingly, the invention is not intended to be limited by the specific disclosures of preferred embodiments herein.

What is claimed is:

1. A wearable body sensor for remotely monitoring a patient, the body sensor comprising:
a case having a skin contact side configured to contact the patient's skin, wherein the case encloses a microcontroller, a battery configured to power the microcontroller, writable memory configured to store data collected by the body sensor, a wireless transmitter in communication with the microcontroller, an impedance sensor in communication with the microcontroller, an electrocardiogram sensor in communication with the microcontroller, a ballistocardiogram sensor in communication with the microcontroller, and a patient orientation sensor in communication with the microcontroller; and
at least two electrodes attached to the skin contact side of the case.

2. The wearable body sensor of claim 1, wherein the case is flexible and water-resistant.

3. The wearable body sensor of claim 1, further comprising a temperature sensor, wherein the temperature sensor is enclosed at least partly by the case.

4. The wearable body sensor of claim 1, further comprising a heart rate sensor, wherein the heart rate sensor is enclosed at least partly by the case.

5. The wearable body sensor of claim 1, further comprising a patient orientation sensor, wherein the patient orientation sensor is enclosed at least partly by the case and comprises an accelerometer.

6. The wearable body sensor of claim 1, further comprising an adhesive disposed on the skin contact side of the case.

7. A remote patient monitoring system, the system comprising:
a wearable body sensor, wherein the body sensor comprises a case having a skin contact side configured to contact the patient's skin, wherein the case encloses a microcontroller, a battery configured to power the microcontroller, writable memory configured to store data collected by the body sensor, a wireless transmitter in communication with the microcontroller, an impedance sensor in communication with the microcontroller, an electrocardiogram sensor in communication with the microcontroller, a ballistocardiogram sensor in communication with the microcontroller, and a patient orientation sensor in communication with the microcontroller, and at least two electrodes attached to the skin contact side of the case; and
a first cellular device in wireless communication with the body sensor.

8. The remote patient monitoring system of claim 7, further comprising a computer in communication with the first cellular device.

9. The remote patient monitoring system of claim 7, further comprising a second cellular device in wireless communication with the computer.
